(19) United States
(12) Patent Application Publication
(10) Pub. No.: US 2025/0381500 A1
(43) Pub. Date: Dec. 18, 2025

(54) SOLVATE AND METHOD OF MANUFACTURING SOLVATE

(71) Applicant: FUJIFILM Corporation, Tokyo (JP)

(72) Inventors: Takafumi NAKAYAMA, Ashigarakami-gun (JP); Shinsuke Tokuoka, Ashigarakami-gun (JP); Naoyuki Morooka, Ashigarakami-gun (JP)

(73) Assignee: FUJIFILM Corporation, Tokyo (JP)

(21) Appl. No.: 18/741,024

(22) Filed: Jun. 12, 2024

Related U.S. Application Data
(63) Continuation of application No. PCT/JP2023/001808, filed on Jan. 20, 2023.

(30) Foreign Application Priority Data
Jan. 28, 2022 (JP) 2022-011665

(51) Int. Cl. B01D 9/00; B01D 15/08; C07D 241/38

(52) U.S. Cl. CPC B01D 9/005; B01D 15/08; C07D 241/38; B01D 2009/0095

(57) ABSTRACT

Provided is a solvate of a compound (A) represented by the following formula and a method of manufacturing the solvate. A solvent forming the solvate includes a cyclic ether compound (B) having a carbon atom, an oxygen atom, and a hydrogen atom, which is 0.05 to 2.0 moles per mole of the compound (A).

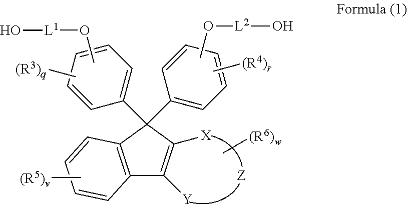

X and Y represent an oxygen atom, a sulfur atom, a nitrogen atom, or a carbon atom,
at least one of X or Y represents a nitrogen atom,
Z represents an atom group that forms a 5- to 7-membered aromatic ring with —X—C═C—Y—,
L¹ and L² represent an alkylene group having 2 to 4 carbon atoms, R³ to R⁶ represent a substituent,
q, r, v, and w represent specific integers,
in a case where v represents an integer of 2 to 4, a plurality of R⁵'s are not linked to each other to form a ring.

### CROSS-REFERENCE TO RELATED APPLICATIONS

This application is a Continuation of PCT International Application No. PCT/JP2023/001808 filed on Jan. 20, 2023, which claims priority under 35 U.S.C. § 119 (a) to Japanese Patent Application No. 2022-011665 filed in Japan on Jan. 28, 2022. Each of the above applications is hereby expressly incorporated by reference, in its entirety, into the present application.

### BACKGROUND OF THE INVENTION

### 1. Field of the Invention

The present invention relates to a solvate and a method of manufacturing a solvate.

### 2. Description of the Related Art

In the related art, a glass material has been used for an optical member of an imaging module such as a camera, a video camera, a mobile phone with a camera, a video phone, or an intercom with a camera. The glass material has optical characteristics suitable for the optical member of the imaging module, can impart desired optical characteristics, and also has excellent environmental tolerance.

However, in the glass material, a reduction in weight and size is not easy, and workability and productivity also deteriorate. On the other hand, a resin cured product can be mass-produced and has excellent workability. Therefore, with a reduction in size of the imaging module in the related art, the resin cured product has been used as an optical member to replace the glass material.

With a reduction in size of the imaging module, a reduction in size is also required for the optical member thereof. However, as the size of the optical member decreases, a problem of chromatic aberration occurs. Accordingly, in an optical member formed of the resin cured product, a configuration of adjusting an Abbe number using a monomer of a curable composition or additives to correct chromatic aberration has been investigated.

For example, WO2017/146022A discloses that a molded body having a sufficiently low Abbe number and high abnormal dispersibility (high Og, F value) can be obtained from a polycarbonate resin that includes a constitutional unit having a heteroatom-containing skeleton similar to a diphenylfluorene skeleton.

In addition, WO2020/184649A describes a solvate of a bisphenol compound having a heteroatom-containing skeleton similar to a diphenylfluorene skeleton associated with a solvent including a specific organic compound having a nitrogen atom. WO2020/184649A describes that, in a case where the purity of a raw material compound is high, a transmittance of a cured product is high, the cured product is preferable as an optical member such as a lens, and the purity of the bisphenol compound is increased up to 96.3 to 98.9% by the solvate described in WO2020/184649A.

### SUMMARY OF THE INVENTION

For a raw material or an intermediate of a resin cured product used as an optical member, it is required to achieve a desired high purity as described in WO2020/184649A. The present inventors have conducted an investigation and found that a diol compound having a heteroatom-containing skeleton similar to a diphenylfluorene skeleton is a non-solvated crystalline form, can be prepared as a high-purity compound having a purity higher than or equal to that of the solvate of the bisphenol compound having a heteroatom-containing skeleton similar to a diphenylfluorene skeleton described in WO2020/184649A, is likely to absorb moisture in the atmosphere depending on crystalline forms, and has high hygroscopicity. For example, in a case where an esterification reaction of a diol compound and (meth)acrylic acid is performed to obtain a monomer for a resin cured product used as an optical member, the high hygroscopicity of the diol compound inhibits the esterification reaction. In a case where a large amount of a reactive substrate is used to increase a reaction rate, the amount of impurities increases, which brings about a decrease in the quality of the resin cured product. This way, from the viewpoint of simultaneously achieving the improvement of the reaction rate and the reduction in the amount of impurities, low hygroscopicity is required for the diol compound having a heteroatom-containing skeleton similar to a diphenylfluorene skeleton.

An object of the present invention is to provide a solvate that is suitable as a raw material compound of a resin forming an optical member such as a lens and a method of manufacturing the solvate.

The above object of the present invention has been achieved by the following means.

<1>

A solvate of a compound (A) represented by Formula (1),
in which a solvent forming the solvate includes a cyclic ether compound (B) having a carbon atom, an oxygen atom, and a hydrogen atom as constituent atoms, and
a ratio of the compound (B) is 0.05 to 2.0 moles per mole of the compound (A),

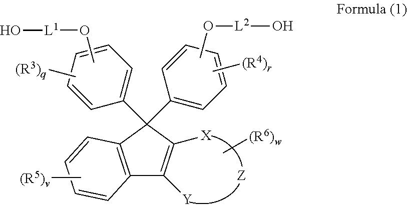

in the formula, X and Y represent an oxygen atom, a sulfur atom, a nitrogen atom, or a carbon atom,
at least one of X or Y represents a nitrogen atom,
Z represents an atom group that forms a 5- to 7-membered aromatic ring with —X—C═C—Y— and is selected from a carbon atom or a heteroatom,
L¹ and L² represent an alkylene group having 2 to 4 carbon atoms,
R³ to R⁶ represent a substituent,
q, r, and v represent an integer of 0 to 4,
w represents an integer of 0 or more and a maximum number of w is a maximum number of substituents that are substitutable with the aromatic ring formed by —X—C═C—Y— and Z, and
in a case where v represents an integer of 2 to 4, a plurality of R⁵'s are not linked to each other to form a ring.
   <2>

The solvate according to <1>,
in which the compound (A) is represented by Formula (2),

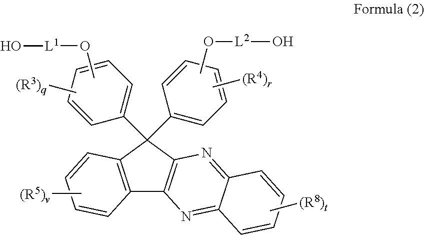

in the formula, L¹, L², R³ to R⁵, q, r, and v have the same definitions as L¹, L², R³ to R⁵, q, r, and v in Formula (1), respectively,
R⁸ represents a substituent, and
t represents an integer of 0 to 4.
   <3>

The solvate according to <1> or <2>,
in which the compound (B) is a tetrahydrofuran compound.
   <4>

A method of manufacturing the solvate according to any one of <1> to <3>, the method comprising:
crystallizing the compound (A) from a solution in which the compound (A) is dissolved in a solvent including the compound (B).
   <5>

The method of manufacturing the solvate according to <4>, comprising:
crystallizing the compound (A) after performing an adsorption treatment step of impurities using an adsorbent on the solution in which the compound (A) is dissolved in the solvent including the compound (B).
   <6>

The method of manufacturing the solvate according to <4> or <5>, comprising:
manufacturing the compound (A) by causing a bisphenol compound represented by Formula (1S) and a cyclic carbonate compound to react with each other in the presence of a basic compound,

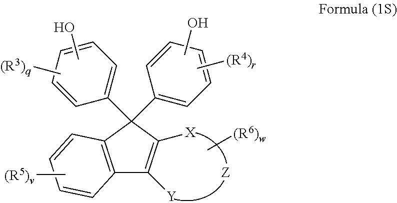

in the formula, R³ to R⁶, X to Z, q, r, v, and w have the same definitions as R³ to R⁶, X to Z, q, r, v, and w in Formula (1), respectively.

In the present invention, in a case where a plurality of substituents or linking groups (hereinafter, referred to as the substituents or the like) represented by a specific symbol or formula are present or in a case where a plurality of substituents or the like are defined at the same time, the respective substituents or the like may be the same as or different from each other (the respective substituents or the like may be the same as or different from each other regardless of the presence of the expression “each independently”) unless otherwise specified. The same can also be applied to the definition of the number of substituents or the like. In addition, in a case where a plurality of substituents and the like are close to each other (particularly adjacent to each other), the substituents and the like may also be linked to each other to form a ring unless otherwise specified. In addition, unless otherwise specified, a ring, for example, an alicyclic ring, an aromatic ring, or a heterocyclic ring may be further fused to form a fused ring.

In the present invention, in a case where an E type and a Z type of a double bond are present in a molecule, any one of the E type or the Z type, or a mixture thereof may be used unless otherwise specified.

In addition, in the present invention, unless otherwise specified, in a case where a compound has one or two or more chiral carbons, for such stereochemistry of chiral carbons, either an (R)-form or an (S)-form can be independently taken. As a result, the compound may be a mixture of optical isomers or stereoisomers such as diastereoisomers or a racemic isomer.

In addition, in the present invention, the expression of a compound includes a compound having a partially changed structure within a range where the effects of the present invention do not deteriorate. Furthermore, a compound which is not specifically described as substituted or unsubstituted may have any substituent within a range where the effects of the present invention do not deteriorate.

In the present invention, a substitute which is not specifically described as substituted or unsubstituted (the same can also be applied to a linking group and a ring) may have any substituent within a range where the desired effects of the present invention do not deteriorate. For example, “alkyl group” represents both an unsubstituted alkyl group and a substituted alkyl group.

In the present invention, in a case where the number of carbon atoms of a certain group is defined, the number of carbon atoms refers to the number of carbon atoms in the entire group unless otherwise specified in the present invention or in the present specification. That is, in a case where this group has a form of further having a substituent, the number of carbon atoms refers to the total number of carbon atoms in the group including this substituent.

In the present invention, a numerical range represented by “to” refers to a range including numerical values described before and after “to” as a lower limit value and an upper limit value.

In the present invention, “(meth)acrylate” represents either or both of acrylate and methacrylate, “(meth)acryloyl” represents either or both of acryloyl and methacryloyl, and “(meth)acrylic acid” represents either or both of acrylic acid and methacrylic acid.

A monomer in the present invention refers to a compound distinguished from an oligomer and a polymer and having a weight-average molecular weight of 1000 or lower.

In the present invention, each of the components including the compound (A) represented by Formula (1), the cyclic ether compound (B), and a solvent other than the cyclic ether compound (B) may be used alone or as a mixture of two or more kinds.

In the present invention, an alkyl group represents a linear or branched alkyl group.

The number of carbon atoms in the alkyl group is preferably 1 to 20, more preferably 1 to 12, and still more preferably 1 to 6.

Examples of the alkyl group include a methyl group, an ethyl group, a propyl group, an isopropyl group, a butyl group, an isobutyl group, a sec-butyl group, a tert-butyl group, a pentyl group, a 1-methylbutyl group, a 3-methylbutyl group, a hexyl group, a 1-methylpentyl group, a 4-methylpentyl group, a heptyl group, a 1-methylhexyl group, a 5-methylhexyl group, a 2-ethylhexyl group, an octyl group, a 1-methylheptyl group, a nonyl group, a 1-methyloctyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group. Among these, a methyl group or an ethyl group is preferable.

The same can also be applied to an alkyl group in a group (an alkoxy group, an alkoxycarbonyl group, an acyl group, an acyloxy group, and the like) including the alkyl group. That is, the description relating to the alkyl group can be applied to the alkyl group in the acyl group and the alkylcarbonyl group and to the alkyl group in the acyloxy group and the alkylcarbonyloxy group.

In addition, the alkyl group may further have a substituent such as a halogen atom or a hydroxy group. Examples of the alkyl group having the substituent include an alkyl halide group and a hydroxyalkyl group.

In the present invention, as an alkenyl group, an alkenyl group having 2 to 6 carbon atoms is preferable, and examples thereof include a vinyl group and an allyl group.

In addition, in the present invention, examples of the alkylene group include a group obtained by removing one hydrogen atom bonded to a terminal carbon atom from the alkyl group. The alkylene group may be a linear alkylene group or a branched alkylene group. Examples of the alkylene group include an ethylene group, a propylene group, and a butylene group.

In the present invention, a structure represented by the following formula shows an isopropylene structure. The isopropylene structure may be any of two structural isomers in which a methyl group is bonded to any one carbon atom forming an ethylene group or in the form of a mixture of the structural isomers.

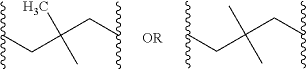

In addition, in the present invention, a structure represented by the following formula shows a butane-1,2-diyl structure (1,2-butylene structure). The butane-1,2-diyl structure (1,2-butylene structure) may be any of two structural isomers in which an ethyl group is bonded to any one carbon atom forming an ethylene group or in the form of a mixture of the structural isomers.

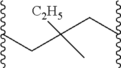 [SMILES: *CC(C)(C*)CC]

In the present invention, a cycloalkyl group denotes a monovalent group obtained by removing any one hydrogen atom from cycloalkane. As the cycloalkyl group, a cycloalkyl group having 3 to 10 carbon atoms is preferable, and examples thereof include a cyclobutyl group, a cyclopentyl group, and a cyclohexyl group.

In the present invention, an aryl group refers to a monovalent group obtained by removing any one hydrogen atom from an aromatic hydrocarbon ring. As the aryl group, an aryl group having 6 to 14 carbon atoms is preferable, and examples thereof include a phenyl group, an 1-naphthyl groups, a 2-naphthyl group, an 1-anthracenyl group, a 2-anthracenyl group, a 9-anthracenyl group, an 1-phenanthryl group, a 2-phenanthryl group, a 3-phenanthryl group, a 4-phenanthryl group, and a 9-phenanthryl group. Among these, a phenyl group is preferable.

In the present invention, a heteroaryl group refers to a monovalent group obtained by removing any one hydrogen atom from an aromatic heterocyclic ring.

An aromatic heterocyclic ring refers to an aromatic ring where a ring is formed by a carbon atom and a heteroatom. Examples of the heteroatom include an oxygen atom, a nitrogen atom, and a sulfur atom. The aromatic heterocyclic ring may be a monocycle or a fused ring, and the number of atoms forming the ring is preferably 5 to 20 and more preferably 5 to 14. The number of heteroatoms in the atoms forming the ring is not particularly limited, but is preferably 1 to 3 and more preferably 1 or 2.

Examples of the heteroaryl group include a furyl group, a thienyl group, a pyrrolyl group, an imidazolyl group, an isothiazolyl group, an isooxazolyl group, a pyridyl group, a pyrazinyl group, a quinolyl group, a benzofuranyl group (preferably, a 2-benzofuranyl group), a benzothiazolyl group (preferably, a 2-benzothiazolyl group), and a benzoxazolyl group (preferably, a 2-benzoxazolyl group).

In the present invention, the alkenyl group, the alkylene group, the cycloalkyl group, the aryl group, the heteroaryl group, the alkoxy group, the alkoxycarbonyl group, and the acyl group may have a substituent as in the alkyl group. Examples of the substituent that may be included include a halogen atom, an acyloxy group, and a cyano group.

In the present invention, examples of the halogen atom include a fluorine atom, a chlorine atom, a bromine atom, and an iodine atom.

In the solvate according to the present invention, the diol compound (A) represented by Formula (1) can be obtained as a solvate having sufficiently suppressed hygroscopicity. In addition, in the solvate according to the present invention, the purity and the colorability satisfy desired levels. By using the solvate according to the present invention as a raw material compound, a resin or a curable monomer forming an optical member such as a lens can be provided as a monomer with high quality, high purity, and low coloration, and the transmittance of the cured product can be improved.

With the manufacturing method according to the present invention, the solvate according to the present invention having sufficiently suppressed hygroscopicity can be manufactured with high purity and high yield.

### BRIEF DESCRIPTION OF THE DRAWINGS

FIG. 1 shows an X-ray diffraction pattern of a crude crystal c1a of a non-solvated compound ci according to Comparative Example 1.

FIG. 2 shows an X-ray diffraction pattern of a crude crystal c1b of the non-solvated compound cl according to Comparative Example 2.

FIG. 3 is an X-ray diffraction pattern of a solvate crystal 1 including, as a solvent, THF (tetrahydrofuran) of a compound c i obtained in Example 1.

### DESCRIPTION OF THE PREFERRED EMBODIMENTS

### <Solvate>

A solvate according to an embodiment of the present invention is a solvate of a compound (A) represented by Formula (1) (hereinafter, also referred to as “compound (A)”), and consists of the compound (A) represented by Formula (1) and a solvent including a cyclic ether compound (B).

In the solvate according to the embodiment of the present invention, the compound (A) represented by Formula (1) and the cyclic ether compound (B) may be used alone or in combination of two or more kinds and are typically used alone.

The present inventors found that hygroscopicity can be sufficiently suppressed for the non-solvated compound represented by Formula (1) by solvating the compound represented by Formula (1) with a solvent including a cyclic ether compound (B) having a carbon atom, an oxygen atom, and a hydrogen atom as constituent atoms. In addition, the solvate according to the embodiment of the present invention is also a compound having reduced coloration and a high purity.

Typically, “solvate” described in the present invention can be isolated as a crystal having a crystal structure different from a crystal consisting of only the compound (A) by allowing the solvent including the cyclic ether compound (B) to be incorporated into the crystalline form of the compound (A), or can also be amorphous. The state or the shape of the solvate according to the embodiment of the present invention is not particularly limited. The solvate according to the embodiment of the present invention is preferably a crystal.

The solvate does not include a compound where a solvent is merely mixed with the compound (A) and can be removed through a solvent removal operation. The solvent removal operation may be an operation that is typically performed. For example, a solvent can be removed by performing drying at 40° C. to 150° C. preferably 60° C. to 120° C. under a reduced pressure (1.0×10³ to 1.0×10⁻¹ Pa) for 1 to 24 hours.

The solvate according to the embodiment of the present invention includes the cyclic ether compound (B) as a solvent at a ratio of 0.05 to 2.0 moles per mole of the compound (A) as described below.

### [Solvent]

In the solvate according to the embodiment of the present invention, a solvent forming the solvate includes a cyclic ether compound (B) having a carbon atom, an oxygen atom, and a hydrogen atom as constituent atoms (hereinafter, also referred to as “compound (B)”).

The compound (B) does not include an atom other than a carbon atom, an oxygen atom, and a hydrogen atom as a constituent atom.

The cyclic ether skeleton in the compound (B) is preferably a 5- to 7-membered ring more preferably a 5- or 6-membered ring. The number of oxygen atoms in the cyclic ether skeleton of the compound (B) is preferably one or two and more preferably one. Any oxygen atom in the cyclic ether skeleton does not form a bond other than an ether bond.

A substituent that may be included in the cyclic ether skeleton of the compound (B) is, for example, preferably an alkyl group or a hydroxy group and more preferably an alkyl group.

Examples of the alkyl group include a branched or linear alkyl group having 1 to 5 carbon atoms. A branched or linear alkyl group having 1 to 3 carbon atoms is preferable, a methyl group or an ethyl group is more preferable, and a methyl group is still more preferable.

Examples of the compound (B) include a tetrahydrofuran compound, a tetrahydropyran compound, a dioxane compound, and a dioxolane compound. A tetrahydrofuran compound, a tetrahydropyran compound, or a dioxane compound is preferable, and a tetrahydrofuran compound is more preferable from the viewpoint of further suppressing hygroscopicity and further improving the purity.

A molecular weight of the compound (B) is preferably 160 or lower, more preferably 140 or lower, and still more preferably 120 or lower. The lower limit value is actually 55 or higher.

Specific examples of the compound (B) include: a tetrahydrofuran compound such as tetrahydrofuran (THF), 2-methyltetrahydrofuran, 2,5-dimethyltetrahydrofuran, 2,2,5,5-tetramethyltetrahydrofuran, or tetrahydrofurfuryl alcohol; a tetrahydropyran compound such as tetrahydropyran or 4-methyltetrahydropyrane; a dioxane compound such as 1,4-dioxane, 1,3-dioxane, or 4-methyl-1,3-dioxane; and a dioxolane compound such as 1,3-dioxolane or 2-methyl-1,3-dioxolane.

Among these, tetrahydrofuran, 1,4-dioxane, or 4-methyltetrahydropyrane is preferable, and tetrahydrofuran is more preferable.

The ratio of the compound (B) in the solvate according to the embodiment of the present invention is 0.05 to 2.0 moles per mole of the compound (A) represented by Formula (1). A preferable range varies depending on the kind of each of the compounds and is not particularly limited. The ratio of the compound (B) per mole of the compound (A) represented by Formula (1) is preferably 0.10 to 1.0 mole, more preferably 0.20 to 0.80 moles, and still more preferably 0.30 to 0.50 moles.

The solvent in the solvate according to the embodiment of the present invention may include an organic compound not having a cyclic ether structure other than the compound (B). Examples of the organic compound not having a cyclic ether structure include an organic compound that is liquid at normal temperature and is generally used as a solvent, and is not particularly limited. Specific examples include those described below regarding a solvent of a manufacturing method (a1).

The ratio of the compound (B) in the solvent forming the solvate according to the embodiment of the present invention may be, for example, 1.0 mass % or more and is preferably 2.5 mass % or more and more preferably 4.0 mass % or more. The upper limit value is not particularly limited and may be 100 mass %.

### [Compound (A) Represented by Formula (1)]

The solvate according to the embodiment of the present invention is a solvate of the diol compound (compound (A)) represented by Formula (1).

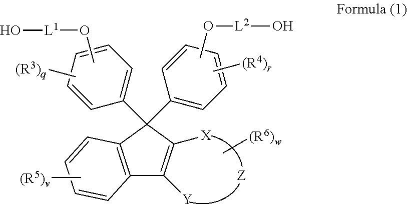

In the formula, X and Y represent an oxygen atom, a sulfur atom, a nitrogen atom, or a carbon atom. At least one of X or Y represents a nitrogen atom. In X and Y, a carbon atom not bonded to R⁶ may be bonded to a hydrogen atom to form CH.

Z represents an atom group that forms a 5- to 7-membered aromatic ring with —X—C═C—Y— and is selected from a carbon atom or a heteroatom. In Z, a carbon atom not bonded to R⁶ may be bonded to a hydrogen atom to form CH.

L¹ and L² represent an alkylene group having 2 to 4 carbon atoms. The above description of the alkylene group can be applied to the alkylene group. L¹ and L² represent preferably an alkylene group having 2 or 3 carbon atoms or more preferably an ethylene group.

From the viewpoint that manufacturing can be performed using the manufacturing method (a1) below and the purity and the transmittance can be further improved, the number of atoms in a portion of L¹ and L² that links —O— and —OH in Formula (1) to each other is preferably 2 or 3 and more preferably 2. Examples of L¹ and L² where the number of atoms in the portion that links —O— and —OH to each other is two include an ethylene group, a propane-1,2-diyl group (1,2-propylene group), and a butane-1,2-diyl group (1,2-butylene group).

R³ to R⁶ represent a substituent.

q, r, and v represent an integer of 0 to 4.

w represents an integer of 0 or more, and a maximum number of w is a maximum number of substituents that are substitutable with the aromatic ring formed by —X—C═C—Y— and Z.

In a case where v represents an integer of 2 to 4, a plurality of R⁵'s are not linked to each other to form a ring.

It is preferable that X and Y represent a nitrogen atom or a carbon atom, and it is more preferable that both X and Y represent a nitrogen atom.

In Formula (1), Z represents preferably an atom group that forms a 5- or 6-membered aromatic ring with —X—C═C—Y— and more preferably an atom group that forms a 6-membered aromatic ring.

In addition, Z represents an atom group selected from a carbon atom or a heteroatom. Preferable examples of the heteroatom that can form Z include an oxygen atom, a sulfur atom, and a nitrogen atom.

Z represents preferably an atom group including at least a carbon atom and selected from a carbon atom and a heteroatom, and more preferably an atom group consisting of a carbon atom.

A substituent that can be used as R³ to R⁶ is not particularly limited, and examples thereof include a halogen atom, an alkyl group, an alkenyl group, an acyl group, an acyloxy group, an alkoxycarbonyl group, a hydroxy group; an alkoxy group, an aryl group, a heteroaryl group, a cycloalkyl group, and a cyano group.

As the substituent that can be used as R³ to R⁶, a halogen atom, an alkyl group, an alkenyl group, an acyl group, an acyloxy group, an alkoxycarbonyl group, an alkoxy group, an aryl group, a heteroaryl group, a cycloalkyl group, or a cyano group is preferable, a halogen atom, an alkyl group, an acyloxy group (alkylcarbonyloxy group), an alkoxy group, an aryl group, or a cyano group is more preferable, a halogen atom, an alkyl group having 1 to 5 carbon atoms, an acyloxy group having 2 to 6 carbon atoms (alkylcarbonyloxy group having 2 to 6 carbon atoms), an alkoxy group having 1 to 5 carbon atoms, a phenyl group, or a cyano group is still more preferable, a halogen atom, a methyl group, an acetyloxy group, a methoxy group, a phenyl group, or a cyano group is still more preferable.

In particular, it is preferable that R³ and R⁴ represent a methyl group or a methoxy group. A plurality of R³'s or a plurality of R⁴'s may form a ring and, in this case, may form a fused ring with a ring to be substituted.

It is preferable that R^{s} represents a halogen atom, a methyl group, or a methoxy group.

R⁶ represents preferably a halogen atom, a methyl group, an acetyloxy group, a methoxy group, a phenyl group, or a cyano group, and more preferably a phenyl group or a cyano group.

In addition, a plurality of R⁶'s represent an alkenyl group and preferably forms a fused ring with the 5- to 7-membered aromatic ring formed by Z and —X—C═C—Y— as described below.

It is preferable that the substituent that can be used as R³ to R⁶ does not have a polymerizable group. The polymerizable group may be a group including any of a vinylidene structure, an oxirane structure, or an oxetane structure. [0041]q, r, and v represent preferably an integer of 0 to 3, more preferably an integer of 0 to 2, and still more preferably 0.

w represents preferably an integer of 0 to 5, more preferably an integer of 0 to 3, still more preferably an integer of 0 to 2, and still more preferably 2.

In a case where q represents an integer of 2 to 4, a plurality of R³'s may be bonded to each other to form a ring, but it is preferable that a plurality of R³'s are not bonded to each other to form a ring. In a case where r represents an integer of 2 to 4, a plurality of R⁴'s may be bonded to each other to form a ring, but it is preferable that a plurality of R⁴'s are not bonded to each other to form a ring.

In a case where v represents an integer of 2 to 4, a plurality of R⁵'s are not linked to each other to form a ring.

In a case where w represents an integer of 2 to 5, a plurality of R⁶'s may be bonded to each other to form a ring. In particular, in a case where w represents an integer of 2 to 5, it is preferable that a plurality of R⁶'s are groups that bonded to each other to form a fused ring with the 5- to 7-membered aromatic ring formed by Z and —X—C═C—Y—. The fused ring may further have a substituent, and preferable examples of the substituent include the substituents described above as the examples of R⁶. Among these, a halogen atom, an alkyl group, an acyl group, an acyloxy group, an alkoxycarbonyl group, a hydroxy group; an alkoxy group, or a cyano group is preferable, a halogen atom, an alkyl group, or an acyloxy group is more preferable, and a halogen atom, a methyl group, or an acetyloxy group is still more preferable.

In a case where a plurality of R⁶'s are bonded to each other to form a fused ring with the 5- to 7-membered aromatic ring formed by Z and —X—C═C—Y—, The number of rings forming the fused ring is preferably 4 or less, more preferably 3 or less, and still more preferably 2. By setting the number of rings forming the fused ring to be in the above-described range, coloration of a cured product including the compound is likely to be suppressed.

In a case where a plurality of R⁶'s are bonded to each other to form a fused ring with the 5- to 7-membered aromatic ring formed by Z and —X—C═C—Y—, this fused ring may be or may not be an aromatic ring but is preferably an aromatic ring.

It is preferable that the compound (A) represented by Formula (1) is a compound represented by Formula (2).

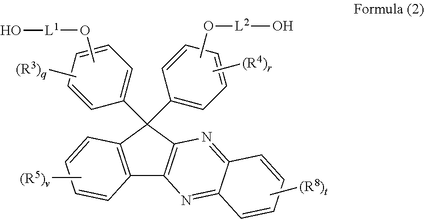

In the formula, L¹, L², R³ to R⁵, q, r, and v have the same definitions as Ll, L², R³ to R⁵, q, r, and v in Formula (1), respectively.

R⁸ represents a substituent.

t represents an integer of 0 to 4.

In a case where a plurality of R⁶'s are bonded to each other to form a fused ring with the 5- to 7-membered aromatic ring formed by Z and —X—C═C—Y—, the description of the substituent that may be included in the fused ring can be applied to a substituent that can be adopted as R⁸.

In a case where t represents an integer of 2 to 4, a plurality of R⁸'s may be linked to each other to form a ring, but it is preferable that a plurality of R's do not form a ring.

R^{I} represents preferably a halogen atom, a methoxycarbonyl group, a methyl group, or a methoxy group, more preferably a halogen atom or a methyl group, and still more preferably a methyl group.

It is preferable that t represents an integer of 0 to 2.

In addition, in Formula (2), it is more preferable that v represents 0 and t represents 1 or 2, and it is still more preferable that v represents 0 and t represents 2. In a case where t represents 1, a substitution site of R⁸ is preferably the 6-position or the 7-position of a formed quinoxaline ring, and in a case where t represents 2, substitution sites of R's are preferably the 6-position and the 7-position of a formed quinoxaline ring.

Hereinafter, preferable specific examples of the compound (A) represented by Formula (1) will be shown below. However, the present invention is not limited to these compounds. In the following compounds, Me represents a methyl group.

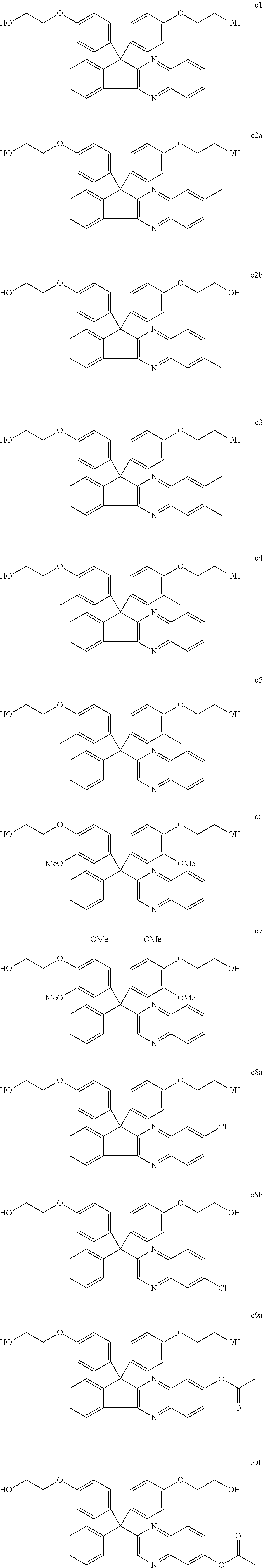

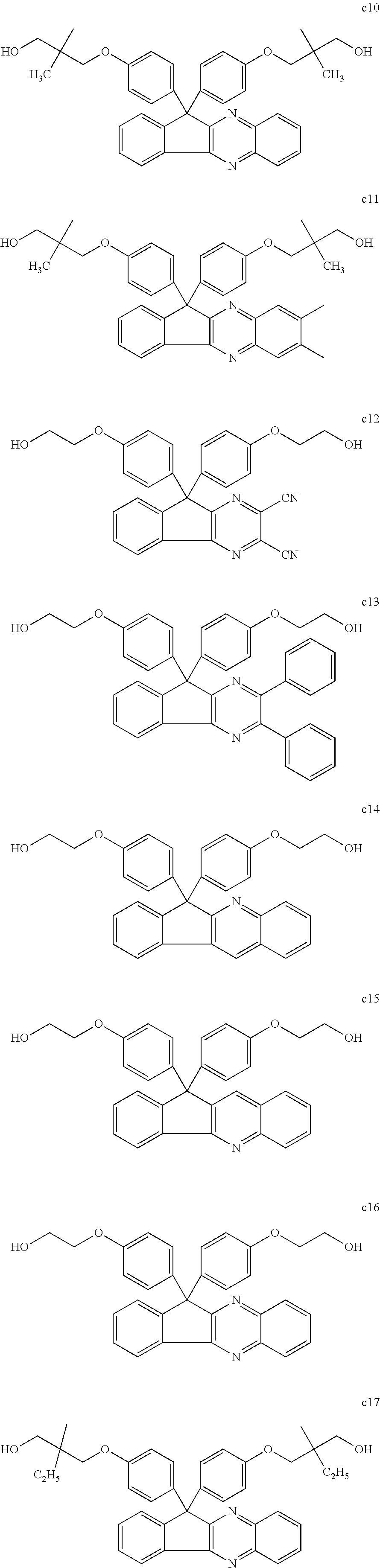

### [Method of Manufacturing Compound (A) Represented by Formula (1)]

The compound (A) represented by Formula (1) can be manufactured using an ordinary method. Examples of the method include the following manufacturing methods (a1) and (a2). From the viewpoint of obtaining the compound (A) having a higher purity and further reduced coloration, the manufacturing method (a1) is preferable for manufacturing the compound (A) represented by Formula (1).

(Manufacturing Method (a1))

In the manufacturing method (a1), the compound (A) can be manufactured by causing a bisphenol compound represented by Formula (1S) and a cyclic carbonate compound to react with each other in the presence of a basic compound.

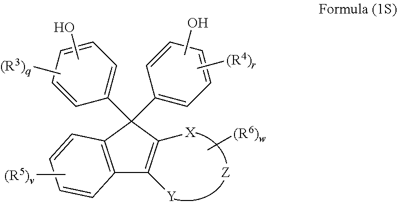

in the formula, R³ to R⁶, X to Z, q, r, v, and w have the same definitions as R³ to R⁶, X to Z, q, r, v, and w in Formula (1), respectively.

As the cyclic carbonate compound, an aliphatic cyclic carbonate compound is preferable. For example, ethylene carbonate, propylene carbonate, trimethylene carbonate, or 1,2-butylene carbonate (4-ethyl-1,3-dioxolane-2-one) can be used, and ethylene carbonate is preferable.

The amount of the cyclic carbonate compound used with respect to 1 mole of the compound represented by Formula (lS) is preferably 2.0 to 10.0 moles, more preferably 2.5 to 8.0 moles, and still more preferably 3.0 to 6.0 moles.

Examples of the basic compound include carbonates, bicarbonates, hydroxides, and organic bases.

Examples of the carbonates include potassium carbonate, sodium carbonate, lithium carbonate, and cesium carbonate.

Examples of the carbonates include potassium bicarbonate, sodium bicarbonate, lithium bicarbonate, and cesium bicarbonate.

Examples of the hydroxides include sodium hydroxide, potassium hydroxide, and lithium hydroxide.

Examples of the organic bases include triethylamine, dimethylaminopyridine, triphenylphosphine, tetramethylammonium bromide, and tetramethylammonium chloride.

Among the basic compound, potassium carbonate or sodium carbonate is suitably used from the viewpoint of excellent handleability.

The basic compound may be used alone or as a mixture of two or more kinds as necessary.

The amount of the basic compound used with respect to 1 mole of the compound represented by Formula (1S) is preferably 0.01 to 1.0 mole, more preferably 0.03 to 0.8 moles, and still more preferably 0.05 to 0.6 moles.

The reaction can also be performed in the presence of or in the absence of a solvent. In particular, from the viewpoints of improving handleability and forming the compound (A) with high yield, it is preferable that the reaction is performed in the presence of the solvent (in the solvent).

As the solvent, a well-known solvent can be used, and the solvent can be appropriately selected depending on the bisphenol compound represented by Formula (lS) or the compound (A) represented by Formula (1). Although not limited thereto, examples of the solvent include: an amide solvent such as N,N-dimethylformamide, N,N-dimethylacetamide, N-methylpyrrolidone, or N-ethylpyrrolidone; an ester solvent such as ethyl acetate, butyl acetate, isobutyl acetate; a ketone solvent such as acetone, methyl ethyl ketone, methyl isobutyl ketone, diisobutyl ketone, or cyclohexanone; an aliphatic nitrile solvent such as acetonitrile or propionitrile; an ether solvent other than the above-described cyclic ether compound (B), such as ethylene glycol dimethyl ether, diethylene glycol dimethyl ether, propylene glycol dimethyl ether, dipropylene glycol dimethyl ether, ethylene glycol monomethyl ether, diethylene glycol monomethyl ether, propylene glycol monomethyl ether, or dipropylene glycol monomethyl ether; a glycol monoether monoacylate solvent such as diethylene glycol monobutyl ether acetate or propylene glycol monomethyl ether acetate; and a hydrocarbon solvent such as xylene or toluene. In particular, from the viewpoint of solubility, an amide solvent is preferable. The solvent can be used alone or in combination of two or more kinds (in the form of a mixed solvent), and may be used in combination with the cyclic ether compound (B).

Reaction conditions of the reaction are not particularly limited. For example, the reaction temperature is preferably 60° C. to 160° C. and more preferably 80° C. to 140° C. The reaction time is preferably 1 to 24 hours and more preferably 2 to 12 hours.

(Manufacturing Method (a2))

In the manufacturing method (a2), the compound (A) can be manufactured by condensing a compound represented by Formula (4) and a compound represented by Formula (5) and/or Formula (6). For example, the manufacturing method (a2) can refer to the description of WO2017/115649A.

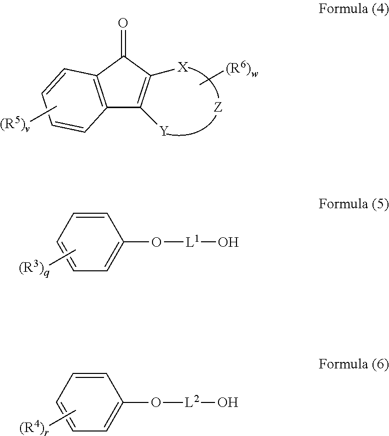

In the formula, L¹, L², R³ to R⁶, X to Z, q, r, v, and w have the same definitions as L¹, L², R³ to R⁶, X to Z, q, r, v, and w in Formula (1), respectively.

It is preferable that the condensation reaction is performed in a solvent including an acid catalyst and a thiol compound.

Examples of the acid catalyst include paratoluene sulfonic acid, methanesulfonic acid, hydrochloric acid, and concentrated sulfuric acid. In addition, as the thiol compound, for example, an alkyl mercaptan such as methyl mercaptan, ethyl mercaptan, n-lauryl mercaptan, or dodecanethiol, or a mercaptocarboxylic acid such as mercaptopropionic acid, thioglycolic acid, or mercaptobenzonic acid can be used.

The description of the solvent in the manufacturing method (a1) can be applied to the solvent that can be used.

The solvent can be used alone or in combination of two or more kinds (in the form of a mixed solvent), and may be used in combination with the cyclic ether compound (B).

Reaction conditions of the reaction are not particularly limited. For example, the reaction temperature is preferably 60° C. to 160° C. and more preferably 80° C. to 140° C. The reaction time is preferably 1 to 24 hours and more preferably 2 to 12 hours. [0065][Method of Manufacturing Solvate]It is preferable that the solvate according to the embodiment of the present invention is manufactured using a crystallization method.

That is, it is preferable that the solvate according to the embodiment of the present invention is manufactured by crystallizing the compound (A) from a solution in which the compound (A) is dissolved in a solvent including the compound (B) (hereinafter, referred to as “the solution of the compound (A)”).

The crystallization method of the compound (A) is not particularly limited. For example, by adding a poor solvent to the compound (A) and/or cooling the solution of the compound (A), the solubility of the compound (A) in the solution decreases to precipitate crystal, and the crystal is separated by filtration. As a result, the solvate according to the embodiment of the present invention can be manufactured.

Even in a case where the solvate according to the embodiment of the present invention cannot be obtained by crystal, an amorphous solvate can be obtained by the crystallization method. The same can be applied to the description relating to the method of manufacturing the solvate described below.

As the solution of the compound (A), a reaction liquid obtained by the manufacturing method (a1) or (a2) (hereinafter, referred to as “reaction liquid”) may be used as it is, or a liquid (hereinafter, referred to as “treatment liquid”) obtained by appropriately performing a liquid separation treatment and/or an adsorption treatment may be used. In addition, after precipitating a crude crystal of the compound (A) from the reaction liquid or the treatment liquid, this crude crystal may be dissolved in the solvent including the compound (B) to prepare the solution of the compound (A). Examples of the solvent that can be used for dissolving the crude crystal are the same as the examples of the solvents described in the manufacturing methods (a1) and (a2).

It is preferable that the compound (B) is used in the solution of the compound (A) at a ratio of 0.10 parts by mass or more with respect to 1 part by mass of the theoretical yield of the compound (A). The upper limit value of the compound (B) is not particularly limited, and it is preferable that the compound (B) is used at a ratio of 20 parts by mass or less with respect to 1 part by mass of the theoretical yield of the compound (A).

As the poor solvent, water or various organic solvents can be used, and hygroscopicity can be further suppressed. In addition, from the viewpoint that the purity and the transmittance can be further improved, an organic solvent is preferable.

Examples of the organic solvent that can be used include an aliphatic acyclic hydrocarbon, an aliphatic cyclic hydrocarbon, an alcohol, and a chain-like ketone. Specifically, examples of the aliphatic acyclic hydrocarbon include hexane and heptane, examples of the aliphatic cyclic hydrocarbon include cyclopentane, cyclohexane, and cycloheptane, examples of the alcohol include methanol, ethanol, 1-propanol, 2-propanol, butanol, pentanol, and octanol, and examples of the chain-like ketone include 2-pentanone, 3-pentanone, 2-hexanone, 3-hexanone, 2-heptanone, and 2-octanone. Among these organic solvents, an aliphatic acyclic hydrocarbon having 5 to 10 carbon atoms is preferable.

The solvate according to the embodiment of the present invention can be manufactured by heating a mixture including the compound (A), the compound (B), and optionally another organic solvent to typically 40° C. or higher and preferably 50° C. or higher, dissolving the compound (A) to prepare the solution of the compound (A) (dissolution step), adding a poor solvent to the compound (A) and/or cooling the solution of the compound (A) to precipitate crystal, and separating the crystal by filtration. The upper limit value of the heating temperature during the heating for dissolving the compound (A) is not particularly limited as long as the solvate according to the embodiment of the present invention can be obtained. The upper limit value of the heating temperature can be appropriately adjusted according to the compound (A), the compound (B), and the other organic solvent that may be optionally included. In order to improve the purity of the obtained solvate and/or the coloration quality (transmittance), it is also preferable that the adsorption treatment is performed using an adsorbent after the dissolution step.

Examples of the adsorbent include: an inorganic adsorbent such as zeolite, alumina, silica gel, activated clay, diatomite earth, or activated carbon; and an organic adsorbent such as a synthetic resin for adsorption or an ion exchange resin. These adsorbents may be used alone or in combination of two or more kinds thereof.

Examples of the inorganic adsorbent that are commercially available include GALLEON EARTH (manufactured by Mizusawa Industrial Chemicals, Ltd.), activated carbons TAIKO S, K, and P (manufactured by Futamura Chemical Co., Ltd.), activated carbon SHIRASAGI A, M, C, and P (manufactured by Osaka Gas Chemicals Co., Ltd.), activated carbon UMEBACHI A, MA, and HC (manufactured by Taihei Chemical Industrial Co., Ltd.), activated carbon GS-A, GS-B, and CL-K (manufactured by Ajinomoto Fine-Techno Co., Inc.), KYOWAAD 200, 500, 600, 700, and 700SEN-S(manufactured by Kyowa Chemical Industry Co., Ltd.), and KW2000 (manufactured by Kyowa Chemical Industry Co., Ltd.), all of which are trade names.

Examples of the synthetic resin for adsorption include DIAION HP10, HP30, and HP2MG and SEPABEADS SP70, SP700, SP825, and SP850 (manufactured by Mitsubishi Chemical Group Corporation), AMBERLITE XAD4, XAD7HP, XAD16HP, and XAD1180 (manufactured by Organo Corporation), and KS and KH (manufactured by Ajinomoto Fine-Techno Co., Inc.), all of which are trade names. Examples of the ion exchange resin include DIAION PK208, PK216, PA306, PA312, WK10, WK20, and CR11 (manufactured by Mitsubishi Chemical Group Corporation), AMBERLITE IRC50, IRC76, IRA400, IRA410, and IRC748 (manufactured by Organo Corporation), and PF, SB, and MA (manufactured by Ajinomoto Fine-Techno Co., Inc.), all of which are trade names.

The adsorption method using the adsorbent is not particularly limited, and a method of directly adding the adsorbent to the solution of the compound (A) and stirring the solution to separate the crystal by filtration may be used, or a method of causing the solution of the compound (A) to pass through a column (adsorption tower) filled with the adsorbent may be used.

### [Use of Solvate]

The compound (A) represented by Formula (1) can be derived into a compound having a side chain with a polymerizable group (group including any of a vinylidene structure, an oxirane structure, or an oxetane structure) using a hydroxy group in the structure of the compound (A), and is useful as an intermediate for manufacturing a curable monomer. For example, the curable monomer can be obtained by an esterification reaction of the compound (A) represented by Formula (1) and a (meth)acrylic acid monomer. As described in WO2017/115649A, a cured product of a composition including the curable monomer derived from the compound represented by Formula (1) can be preferably used as an optical member such as a lens.

In addition, the compound (A) represented by Formula (1) can be used as it is as one component of the curable composition for manufacturing an optical member such as a lens. For example, by using the diol compound (A) represented by Formula (1) as a monomer, a polycarbonate resin can also be manufactured with, for example, a method described in WO2017/146022A.

As described above, with the compound (A) represented by Formula (1), the solvate with the solvent including the cyclic ether compound (B) can be obtained as a solvate where hygroscopicity is sufficiently suppressed and purity and colorability satisfy a desired level. Therefore, a resin or a curable monomer forming an optical member such as a lens can be provided with high quality, high purity, and low coloration.

### EXAMPLES

Hereinafter, the present invention will be described in more detail on the basis of examples. Materials, used amounts, ratios, treatment details, treatment procedures, and the like shown in the following examples can be appropriately changed within a range not departing from the scope of the present invention. Therefore, the scope of the present invention should not be construed as being limited to the following specific examples.

MeOH represents methanol, DMSO represents dimethyl sulfoxide, THF represents tetrahydrofuran, and DMAc represents N,N-dimethylacetamide. The room temperature means 25° C.

w/v % represents a mass per volume % concentration.

A ratio in a mixed liquid is represented by volume.

Vacuum drying at 100° C. represents drying for 8 hours under conditions of 100° C. and 100 to 200 Pa.

### SYNTHESIS EXAMPLES

### Synthesis Example 1: Synthesis of Compound c1 with Manufacturing Method (a1) Described Above

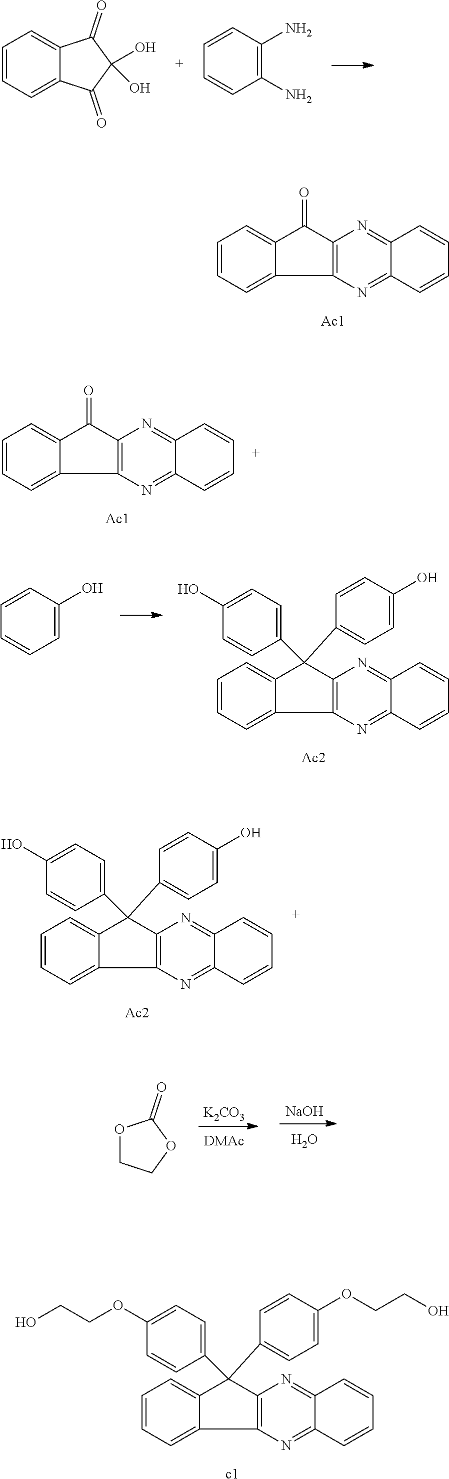

An intermediate Ac1 was synthesized using the same method as that of paragraph [0080] of JP6712633B. Next, an intermediate Ac2 was synthesized using the same method as that of paragraph [0083] of JP6712633B.

200 g of the intermediate Ac2, 176 g of ethylene carbonate, 34.6 g of potassium carbonate, and 176 g of N,N-dimethylacetamide were weighed into a 2 L three-neck flask equipped with a cooling pipe, and were caused to react with each other in an oil bath at 130° C. for 3 hours. After the reaction, the solution was cooled to 80° C. or lower, and 80 mL of water and 160 mL of a 50 w/v % sodium hydroxide aqueous solution were added and were caused to react with each other for 1 hour. The solution was cooled to 50° C. or lower, 500 mL of water and 500 mL of MeOH were added, and precipitated crystal was collected by filtration. This crystal was cleaned with water and was vacuum-dried at 100° C. to obtain 210 g of a crude crystal c1a of a compound cl. In ¹H-NMR measurement (400 MHz, DMSO-d₆), both of the contents of N,N-dimethylacetamide and methanol were less than 1 mole with respect to 100 moles of the compound ci.

### Synthesis Example 2: Synthesis of Compound cl with Manufacturing Method (a2) Described Above

150 g of the intermediate Ac1, 450 g of 2-phenoxyethanol, 225 mL of toluene, 180 mL of methanesulfonic acid, and 2.4 mL of dodecanethiol were weighed into a 2 L three-neck flask equipped with a cooling pipe, and were caused to react with each other in an oil bath at 150° C. for 2 hours. After the reaction, after cooling the solution to room temperature, an upper layer of an organic layer separated into two layers was removed, 3 L of ethyl acetate and water were added, and the solution was stirred. Precipitated crystal was collected by filtration and was recrystallized by a mixed solvent including ethyl acetate and hexane. Next, the crystal was collected by filtration. This crystal was vacuum-dried at 100° C. to obtain 160 g of a crude crystal c1b of the compound cl. In ¹H-NMR measurement (400 MHz, DMSO-d₆), both of the contents of ethyl acetate and hexane were less than 1 mole with respect to 100 moles of the compound c1.

### Synthesis Example 3: Synthesis of Compound c3 with Manufacturing Method (a1) Described Above

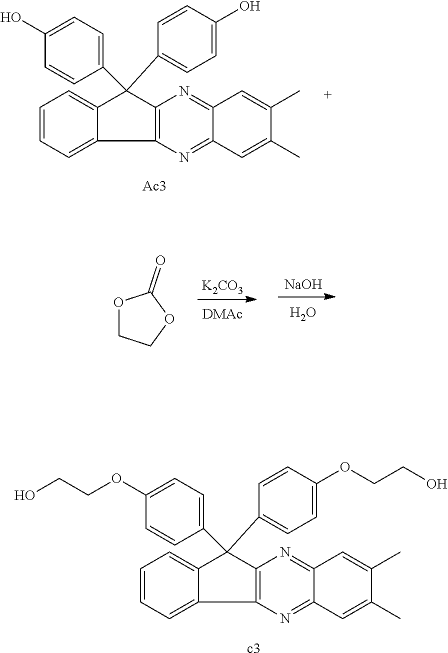

Using the same method as that of paragraphs [0066], [0067], and [0072] of WO2020/184649A, a compound Ac3 (solvate with N,N-dimethylacetamide) was obtained. Next, a crude crystal c3 of the compound c3 was obtained using the same method as that of Synthesis Example 1, except that the compound Ac3 was used instead of the compound Ac2. In ¹H-NMR measurement (400 MHz, DMSO-d₆), both of the contents of N,N-dimethylacetamide and methanol were less than 1 mole with respect to 100 moles of the compound c3.

This way, the crude crystals c1a, c1b, and c3 obtained in Synthesis Examples 1 to 3 were not solvated, in which the contents of the solvents in the obtained crude crystals were less than 1 mole with respect to 100 moles of the compound (A).

### EXAMPLES

### Example 1

20 g of the crude crystal c1a and 140 mL of tetrahydrofuran (THF) were added to a 1 L three-neck flask equipped with a cooling pipe, and the solution was stirred at 60° C. for 1 hour to dissolve the crude crystal. Next, 1.0 g of activated carbon SHIRASAGI A (trade name, manufactured by Osaka Gas Chemicals Co., Ltd.) as an adsorbent and 2.0 g of KYOWAAD 700SEN-S(trade name, manufactured by Kyowa Chemical Industry Co., Ltd.) were added, the solution was stirred at 60° C. for 2 hours, and the adsorbent was separated by celite filtration. The obtained filtrate was concentrated up to 80 g by an evaporator set to 45° C., 15 mL of hexane as a poor solvent was added, and the solution was stirred. After precipitating crystal, the solution was further stirred in an ice bath for 1 hour. The precipitated crystal was collected by filtration, was cleaned with 50 mL of a mixed liquid including THF and hexane at a ratio of 1v:1v (representing 1:1 by volume), and was vacuum-dried at 100° C. As a result, 18.0 g of a solvated crystal 1 of the compound cl and THF was obtained. In ¹H-NMR measurement (400 MHz, DMSO-d₆) of the solvated crystal 1, the content of THF was 35 moles with respect to 100 moles of the compound c i.

### Example 2

16.5 g of a solvated crystal 2 of the compound c1 and 1,4-dioxane was obtained using the same method as that of Example 1, except that THF was changed to 1,4-dioxane. In ¹H-NMR measurement (400 MHz, DMSO-d₆) of the solvated crystal 2, the content of 1,4-dioxane was 40 moles with respect to 100 moles of the compound c1.

### Example 3

18.5 g of a solvated crystal 3 of the compound cl and 4-methyltetrahydropyrane was obtained using the same method as that of Example 1, except that THF was changed to 4-methyltetrahydropyrane. In ¹H-NMR measurement (400 MHz, DMSO-d₆) of the solvated crystal 3, the content of 4-methyltetrahydropyrane was 50 moles with respect to 100 moles of the compound ci.

### Example 4

18.4 g of a solvated crystal 4 of the compound ci and THF was obtained using the same method as that of Example 1, except that an adsorption treatment of impurities using activated carbon SHIRASAGI A and KYOWAAD 700SEN-S was not performed. In ¹H-NMR measurement (400 MHz, DMSO-d₆) of the solvated crystal 4, the content of THF was 41 moles with respect to 100 moles of the compound c i.

### Example 5

17.0 g of a solvated crystal 5 of the compound ci and THF was obtained using the same method as that of Example 1, except that the crude crystal c1a was changed to the crude crystal c1b and KYOWAAD 700SEN-S used as the adsorbent was changed to KYOWAAD 500SN (trade name, manufactured by Kyowa Chemical Industry Co., Ltd.). In ¹H-NMR measurement (400 MHz, DMSO-d₆) of the solvated crystal 5, the content of THF was 35 moles with respect to 100 moles of the compound c l.

### Example 6

17.5 g of a solvated crystal 6 of the compound cl and THF was obtained using the same method as that of Example 5, except that an adsorption treatment of impurities using activated carbon SHIRASAGI A and KYOWAAD 500SN was not performed. In ¹H-NMR measurement (400 MHz, DMSO-d₆) of the solvated crystal 5, the content of THF was 35 moles with respect to 100 moles of the compound c l.

### Example 7

15.5 g of a solvated crystal 7 of the compound c3 and THF was obtained using the same method as that of Example 1, except that the crude crystal c1a was changed to the crude crystal c3. In ¹H-NMR measurement (400 MHz, DMSO-d₆) of the solvated crystal 7, the content of THF was 33 moles with respect to 100 moles of the compound c3.

### Example 8

13.9 g of a solvated crystal 8 of the compound ci and THF was obtained using the same method as that of Example 1, except that the solvent in which the crude crystal c1a was dissolved was changed from 140 mL of THF to a mixed solvent including 30 mL of THF and 70 mL of N,N-dimethylacetamide. In ¹H-NMR measurement (400 MHz, DMSO-d₆) of the solvated crystal 8, the content of THF was 25 moles with respect to 100 moles of the compound c i.

### Example 9

17.3 g of a solvated crystal 9 of the compound ci and THF was obtained using the same method as that of Example 1, except that 40 mL of water was used as the poor solvent instead of 15 mL of hexane. In ¹H-NMR measurement (400 MHz, DMSO-d₆) of the solvated crystal 9, the content of THF was 14 moles with respect to 100 moles of the compound c I.

### Comparative Examples 1 to 3

The crude crystal c1a, c1b, and c3 obtained in Synthesis Examples 1 to 3 were used as compounds of Comparative Examples 1 to 3 for the following evaluations, respectively.

### [Evaluation 1: Hygroscopicity]

1.0 g of each of the crystals that was vacuum-dried at 100° C. was accurately weighed into a 10 mL vial container, and was left to stand for 24 hours in a high-temperature and high-humidity bath set to 30° C. and 80% RH (relative humidity). A mass increase ratio (water absorption ratio) of the crystal before and after 24 hours was evaluated for hygroscopicity based on the following standards. As the water absorption ratio decreases, the hygroscopicity of the crystal is suppressed. Table 1 collectively shows the results.

### —Evaluation Standards—

A: less than 1.0 mass %
B: 1.0 mass % or more and less than 2.0 mass %
C: 2.0 mass % or more and less than 3.0 mass %
D: 3.0 mass % or more

TABLE 1

| | Example 1 | Example 2 | Example 3 | Example 4 | Example 5 | Example 6 |
|---|---|---|---|---|---|---|
| | Solvated Crystal 1 | Solvated Crystal 2 | Solvated Crystal 3 | Solvated Crystal 4 | Solvated Crystal 5 | Solvated Crystal 6 |
| Compound (A) | c1 | c1 | c1 | c1 | c1 | c1 |
| Solvent forming | THF | 1,4-dioxane | 4-methyltetra- | THF | THF | THF |
| Solvate | | | hydropyrane | | | |
| (B)/(A) | 0.35 | 0.40 | 0.50 | 0.41 | 0.35 | 0.35 |
| Synthesis Route | (a1) | (a1) | (a1) | (a1) | (a2) | (a2) |
| of Compound (A) | | | | | | |
| Adsorbent | Activated Carbon | Activated Carbon | Activated Carbon | — | Activated Carbon | — |
| | SHIRASAGI A | SHIRASAGI A | SHIRASAGI A | | SHIRASAGI A | |
| | KYOWAAD 700 | KYOWAAD 700 | KYOWAAD 700 | | KYOWAAD 500 | |
| Hygroscopicity | A | B | A | A | A | A |

| | | | | Comparative | Comparative | Comparative |
|---|---|---|---|---|---|---|
| | Example 7 | Example 8 | Example 9 | Example 1 | Example 2 | Example 3 |
| | Solvated Crystal 7 | Solvated Crystal 8 | Solvated Crystal 9 | Crude Crystal c1a | Crude Crystal c1b | Crude Crystal c3 |
| Compound (A) | c3 | c1 | c1 | c1 | c1 | c3 |
| Solvent forming | THF | THF | THF | — | — | — |
| Solvate | | | | | | |
| (B)/(A) | 0.33 | 0.25 | 0.14 | — | — | — |
| Synthesis Route | (a1) | (a1) | (a1) | (a1) | (a2) | (a1) |
| of Compound (A) | | | | | | |
| Adsorbent | Activated Carbon | Activated Carbon | Activated Carbon | — | — | — |
| | SHIRASAGI A | SHIRASAGI A | SHIRASAGI A | | | |
| | KYOWAAD 700 | KYOWAAD 700 | KYOWAAD 700 | | | |
| Hygroscopicity | A | B | C | D | D | D |

(Notes in table)
Compound (A)
c1 and c3: c1 and c3 described above in the specific examples of the compound (A)
Synthesis Route of Compound (A)
(a1) and (a2): Manufacturing methods (a1) and (a2) of the compounds described above
Adsorbent(s)
Activated carbon SHIRASAGI A: trade name, manufactured by Osaka Gas Chemicals Co., Ltd.
KYOWAAD 700: KYOWAAD 700SEN-S (trade name), manufactured by Kyowa Chemical Industry Co., Ltd.
KYOWAAD 500: KYOWAAD 500SN (trade name), manufactured by Kyowa Chemical Industry Co., Ltd.

(B)/(A) represents a molar ratio of the cyclic ether compound (B) in the solvent forming the solvate to the compound (A).

In Comparative Examples 1 to 3, the crude crystals c1a, c1b, and c3 were not solvated, and thus the fields of the solvent forming the solvate and (B)/(A) are shown as “-”.

“-” in the field of the adsorbent(s) shows that the adsorption treatment of impurities using the adsorbent(s) was not performed.

As shown in Table 1, all of the non-solvated compounds cl and c3 (the crude crystals c1a, c1b, and c3) according to Comparative Examples 1 to 3 had a high water absorption ratio and showed high hygroscopicity. On the other hand, all of the compound cl (solvated crystals 1 to 6, 8, and 9) according to Examples 1 to 6, 8, and 9 and the compound c3 (solvated crystal 7) according to Example 7 as solvates had a low water absorption ratio and showed sufficiently suppressed hygroscopicity.

Regarding the solvated crystals 1 to 9 according to Examples 1 to 9, the HPLC (high-performance liquid chromatography) purity and the transmittance were measured and calculated as follows.

### [Evaluation 2: HPLC Purity]

Using a high-performance liquid chromatograph (SPD-10AV VP) (manufactured by Shimadzu Corporation), analysis was performed under the following conditions, the purity of the crystal was measured based on the peak surface area and was evaluated based on the following standards. Further, in a case where the crystal was solvated, the HPLC purity was calculated after excluding the peak derived from the solvent. The results are summarized in Table 2.

### (Measurement Conditions)

Column: TSKgel ODS-100Z, 5 m (4.6 mmφ×150 mm) (manufactured by Tosoh Corporation)
Column temperature: 40° C.
Eluent: a mixed liquid where acetonitrile:pure water:phosphoric acid (volume ratio)=500:500:1 was used
Flow rate: 1.0 ml/min
Detection wavelength: 254 nm
Injection volume: 10 L
Sample: a solution in which the crystal was dissolved in the eluent such that the concentration thereof reached 0.20 mg/ml was used

### —Evaluation Standards—

A: 99.5% or more
B: 99.0% or more and less than 99.5%
C: 98.0% or more and less than 99.0%
D: less than 98.0%

### [Evaluation 3: Transmittance]

The transmittance of the dilute solution of the crystal at a wavelength of 420 nm was measured under the following conditions using a spectrophotometer (trade name: UV-2550, manufactured by Shimadzu Corporation), and was evaluated based on the following standards. As the transmittance at 420 nm increases, yellow coloration of the crystal decreases. Table 2 collectively shows the results.

### (Measurement Conditions)

Cell: square quartz cell (optical path length: 1 cm)
Sample: a solution in which the crystal was dissolved in tetrahydrofuran (THF) such that the concentration thereof reached 50.0 mg/50 ml was used
Blank: THF

### —Evaluation Standards—

A: 99.5% or more
B: 99.0% or more and less than 99.5%
C: 98.0% or more and less than 99.0%
D: less than 98.0%

TABLE 2

| | Example | Example | Example | Example | Example | Example | Example | Example | Example |
|---|---|---|---|---|---|---|---|---|---|
| | 1 | 2 | 3 | 4 | 5 | 6 | 7 | 8 | 9 |
| HPLC Purity | A | A | B | A | B | B | A | A | B |
| Transmittance | A | A | A | B | B | C | A | A | B |

As shown in Table 2, all of the compound cl (solvated crystals 1 to 6, 8, and 9) according to Examples 1 to 6, 8, and 9 and the compound c3 (solvated crystal 7) according to Example 7 had a purity of 99.0% or more and a transmittance of 98.0% or more and satisfied a desired high purity and low colorability.

### <XRD Measurement>

In order to verify that the crystalline forms of the crystal obtained in Examples of the present invention and the crystal obtained in Comparative Examples were different from each other, powder X-ray diffraction (XRD) measurement was performed under the following measurement conditions. As an XRD device, a well-known device can be used. For example, the measurement was performed using SmartLab (manufactured by Rigaku Corporation), D8 Discover (manufactured by Bruker Corporation), or Empyrean (manufactured by Malvern Panalytical Ltd.), all of which are trade names.

### —Measurement Conditions—

Tube: CuKα
Output: 40 kV 30 mA
Measurement range: 2θ=2° to 60°
Sampling interval: 0.020
Measurement rate: 5°/min

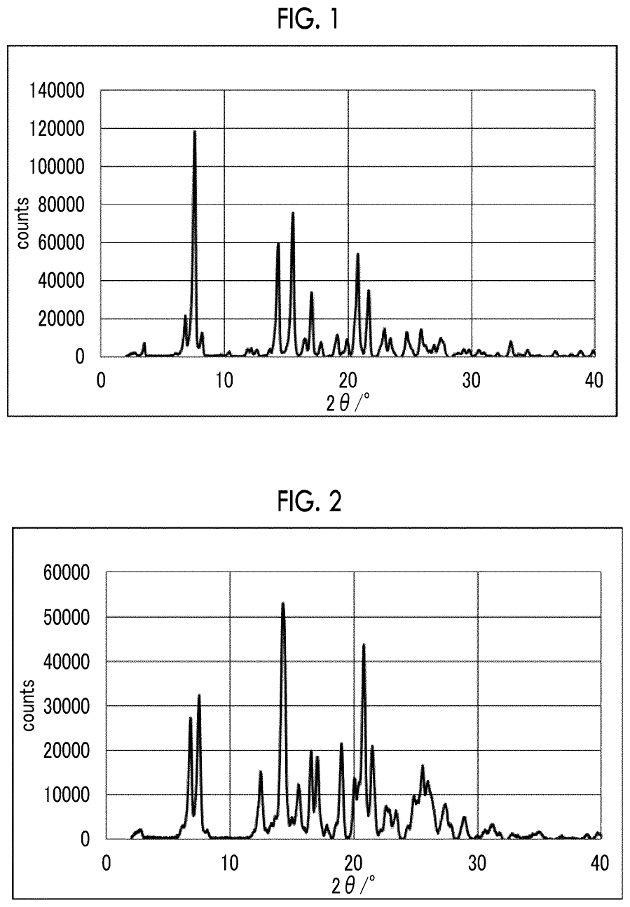

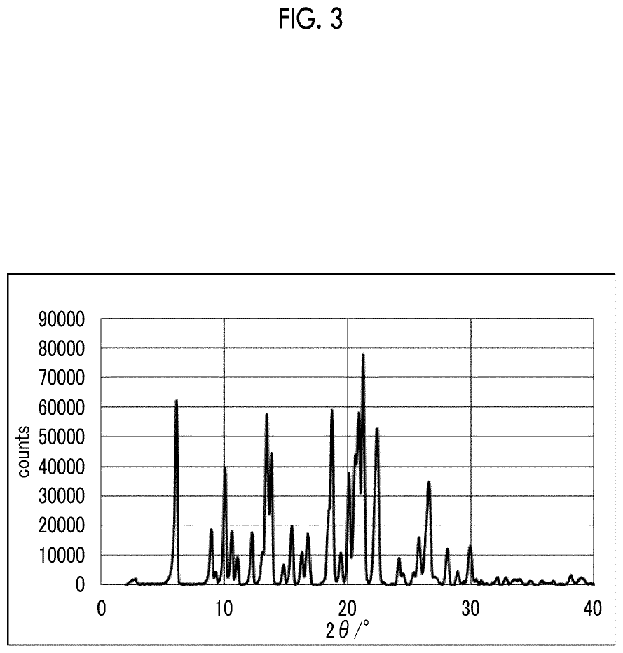

FIGS. 1 and 2 show the XRD measurement results of the compound cl (crude crystals c1a and c1b) according to Comparative Examples 1 and 2 that were not the solvates with the cyclic ether compound. FIG. 3 shows the XRD measurement result of the compound cl (solvated crystal 1) according to Example 1 that was the solvate with the cyclic ether compound (B). In all of FIGS. 1 to 3, a diffraction pattern was shown at a diffraction angle 2θ=40° where a characteristic peak was shown.

From a comparison between FIGS. 1 and 2 (Comparative Examples 1 and 2) and FIG. 3 (Example 1), it can be seen that, in the solvated crystal 1 that was the solvate with the cyclic ether compound, a profile that was different from the powder X-ray diffraction measurement results of the crude crystals c1a and c1b for Comparison that were not the solvates was shown, and the crystalline form was different.

The present invention has been described using the embodiments. However, unless specified otherwise, any of the details of the above description is not intended to limit the present invention and can be construed in a broad sense within a range not departing from the concept and scope of the present invention disclosed in the accompanying claims.

What is claimed is:

1. A solvate of a compound (A) represented by Formula (1),
wherein a solvent forming the solvate includes a cyclic ether compound (B) having a carbon atom, an oxygen atom, and a hydrogen atom as constituent atoms, and
a ratio of the compound (B) is 0.05 to 2.0 moles per mole of the compound (A), 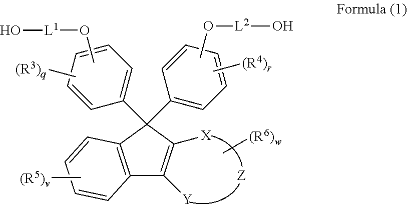
in the formula, X and Y represent an oxygen atom, a sulfur atom, a nitrogen atom, or a carbon atom,
at least one of X or Y represents a nitrogen atom,
Z represents an atom group that forms a 5- to 7-membered aromatic ring with —X—C═C—Y— and is selected from a carbon atom or a heteroatom,
L¹ and L² represent an alkylene group having 2 to 4 carbon atoms,
R³ to R⁶ represent a substituent,
q, r, and v represent an integer of 0 to 4,
w represents an integer of 0 or more and a maximum number of w is a maximum number of substituents that are substitutable with the aromatic ring formed by —X—C═C—Y— and Z, and
in a case where v represents an integer of 2 to 4, a plurality of R⁵'s are not linked to each other to form a ring.

2. The solvate according to claim 1,
wherein the compound (A) is represented by Formula (2), 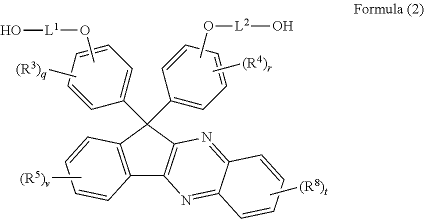
in the formula, L¹, L², R³ to R⁵, q, r, and v have the same definitions as L¹, L², R³ to R⁵, q, r, and v in Formula (1), respectively,
R⁸ represents a substituent, and
t represents an integer of 0 to 4.

3. The solvate according to claim 1,
wherein the compound (B) is a tetrahydrofuran compound.

4. A method of manufacturing the solvate according to claim 1, the method comprising:
crystallizing the compound (A) from a solution in which the compound (A) is dissolved in a solvent including the compound (B).

5. The method of manufacturing the solvate according to claim 4, comprising:
crystallizing the compound (A) after performing an adsorption treatment step of impurities using an adsorbent on the solution in which the compound (A) is dissolved in the solvent including the compound (B).

6. The method of manufacturing the solvate according to claim 4, comprising:
manufacturing the compound (A) by causing a bisphenol compound represented by Formula (1S) and a cyclic carbonate compound to react with each other in the presence of a basic compound, 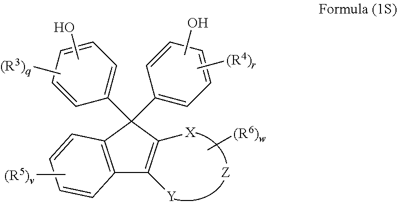
in the formula, R³ to R⁶, X to Z, q, r, v, and w have the same definitions as R³ to R⁶, X to Z, q, r, v, and w in Formula (1), respectively.
